(19) United States
(12) Patent Application Publication
(10) Pub. No.: US 2004/0224321 A1
(43) Pub. Date: Nov. 11, 2004

(54) Micro/nano-structures fabricated by laser ablation for micro-array applications

(75) Inventors: Dan Veniamin Nicolau, Balwyn North (AU); Elena Ivanova, Lower Templestowe (AU); Jonathan Wright, Kew East (AU); Duy Pham, Vermont South (AU)

Correspondence Address: SUGHRUE MION, PLLC, 2100 Pennsylvania Avenue, NW, Washington, DC, 20037-3213, US

(73) Assignee: SWINBURNE UNIVERSITY OF TECHNOLOGY

(21) Appl. No.: 10/689,738

(22) Filed: Oct. 22, 2003

(30) Foreign Application Priority Data
Oct. 31, 2002 (AU) 2002952384

(51) Int. Cl. C12Q 1/68

(52) U.S. Cl. 435/6; 436/518

(57) ABSTRACT

Micro/nano-structures fabricated by laser ablation and suitable for use in micro-arrays and micro-assays are described. In particular, micro-structures having combinatorial surfaces that allow molecules to attach to a localised area of the surface according to the characteristics of the localised area. Methods of fabricating such micro/nano-structures and their use in micro-arrays and micro-assays is also described.

### FIELD OF THE INVENTION

The present invention relates to structures suitable for use in arrays, and in particular but not exclusively, to structures having combinatorial surfaces that allow molecules to attach to a localised area of the surface according to the characteristics of the localised area. The invention also relates to methods of fabricating structures having combinatorial surfaces and to their use in arrays and assays.

### BACKGROUND OF THE INVENTION

Micro-structures are fabricated and used in DNA microarrays and microassays which may provide a rapid and moderate cost biosensing system, for example, for detecting DNA base-pairing or hybridisation. Microarrays are orderly arrangements of samples deposited on a micro-structure. The typical size of a sample spot in a microarray is in the range of tens of microns to a few hundred, microns, however, sample spots written with AFM may have a size in the order of nanometers. Each microarray may hold hundreds of thousands of samples.

Technologies available for the fabrication of these structures must ensure the confinement of different sample molecules in localised areas, which may be flat or profiled. Technologies that are available for fabricating these structures include (i) spotted-array-based methods, De Wildt et al. (2000), Walter et al. (2000); (ii) soft lithography, Zhao et al. (1997), Bernard et al. (1998) (iii) photolithography, Fodor et al. (1991), U.S. Pat. No. 5,391,463, Nicolau et al. (1998), Nicolau et al. (1999); (iv) scanning probe lithography, Wadu-Mesthrige et al. (1999); (v) laser or ion-beam ablation, Schwarz et al. (1998), U.S. Pat. No. 5,858,801, and (vi) microfabrication of profiled features for e.g. microfluidic devices, Wang et al. (2000), Sundberg (2000), Nicolau and Cross (2000), McDonald et al. (2001), Ismagilov et al. (2001). These methods have been listed, not comprehensively, in the order of their ‘3D-ness’, that is, starting with features that are elevated above the surface by a few nanometers (methods i, ii, and iii); to quasi-flat features (methods iv and v); and ending with samples that are placed on the bottom of etched or developed micro-features (methods v and vi). While some types of biodevices dictate a particular design of the biodevices (eg. microfluidics devices require profiled channels) others do not (eg. microarrays normally have a flat surface). The profiled features of methods (v) and (vi) have the advantage of minimization of inter-spot contamination and the drawback of difficult access of the recognition component (eg. antigen for antibody microarray) in a micro-defined area.

Two potentially important surface-related problems of this technology are (i) the possible difference between the surface concentration of different molecules on the same surface and (ii) the possible surface-induced denaturation of the structure and subsequently the change of the bioactivity of the adsorbed biomolecules, Andrade and Hlady (1991).

Economic requirements dictate the preference for use of a minimum amount of material for fabrication and operation of the micro-assay. While classical microarray technology involves flat surfaces, with inherent spread of the small volume of the analyte solution in the deposited droplet, a profiled microfabricated location in which the droplet is deposited would be a more efficient solution. However, the depth of the profiled feature has to be minimized in order to allow free diffusion of the recognition molecule in the micro-fabricated well.

Among the many procedures for microfabrication, ablation has the advantage of a step-wise process without the involvement of fluids such as in microlithography. In principle, there are few possibilities to fabricate the micro-wells, each of which have advantages and drawbacks. A first possibility is the ablation of a protein-blocked single layer of a polymer, Schwarz et al. (1998), U.S. Pat. No. 5858801, which is preferably designed to promote molecule adsorption, especially proteins, without surface-induced denaturation. This is the simplest choice. However, this approach requires either expensive laser ablation tools operating in deep-UV (e.g. 248 nm) and non-fluorescent polymers (e.g. PMMA), or the use of more convenient (e.g. near-UV) lasers and polymers that absorb in that region, but which are likely to interfere with the detection through background fluorescence. A second possibility is adopting a bilayer structure with an ablatable layer on the bottom and a molecule-adsorbing, sacrificial layer on top. However, experiments proved that the ablated material (e.g. Au) can not be efficiently released during the ablation through the top polymeric layer, which leads to the frequent peel-off of large areas of the bilayer structure. A third possibility is to deposit a very thin ablatable layer on top of a molecule-adsorbing, transparent to laser wavelength, non-ablatable polymeric layer. This technological avenue raises the issue of the fate of the physics and chemistry of the top surface of the bottom layer, which is exposed to large amounts of energy during the ablation of the top layer. The logical approach would be to tune the ablation in a manner that will preserve the bottom layer. Unfortunately there is only a remote possibility that this can be achieved.

Among the enabling technologies for the above patterning methods, laser beams are capable, according to the exposure energy and the sensitivity or absorbance of the exposed material, to enable both photolithography and photo-assisted etching. Also, focused laser beams can, in principle, solve a critical fabrication and operating problem of the structures better than most other alternative methods, i.e. they may provide controlled and confined variation of the surface properties of the areas upon which different molecules are adsorbed.

Advantageously, one embodiment of the present invention may provide the fabrication via laser ablation of shallow-profiled structures with surfaces having areas tailored to accommodate an universal adsorption of molecules.

A further problem associated with the use of arrays is the identification of different samples within the array, or the identification of different test samples that are applied to the array in an assay. Advantageously, at least one embodiment of the present invention provides an ‘informationally-addressable’ structure or an array where information about each sample in the array or each test sample applied to the array in a assay is encoded by the combination of shallow-profiled features within the array.

### SUMMARY OF THE INVENTION

According to one aspect of the present invention there is provided a structure comprising (i) a first layer comprising a molecule-adsorbing, substantially non-ablatable material,-and (ii) a second layer comprising an ablatable material, wherein the second layer is disposed on the first layer and wherein at least a portion of the second layer has been ablated to expose a surface of first layer and form at least one profiled feature.

Preferably the exposed surface of the first layer comprises at least two localized areas having molecule-adsorbing capacities for molecules with different adsorbing properties. In another preferred embodiment, a plurality of portions are ablated to form an informationally-addressable pattern.

According to another aspect of the invention there is provided an array comprising (a) a micro-structure which comprises (i) a first layer comprising a molecule-adsorbing, substantially non-ablatable material, and (ii) a second layer of ablatable material, wherein the second layer is disposed on the first layer and a plurality of portions of the second layer have been ablated to expose a surface of the first layer and thereby form a plurality of profiled features, and (b) at least one biomolecule adsorbed on the surface of the first layer in at least one of the plurality of profiled features.

In a further aspect, the present invention provides a method of fabricating a structure as described above, comprising the steps of:

(a) obtaining a substrate supporting (i) a first layer comprising a molecule-adsorbing, substantially non-ablatable material and (ii) a second layer comprising a ablatable material disposed on the first layer;

(b) laser ablating at least a portion of the second layer to expose a surface of the first layer to form at least one profiled feature.

In yet a further aspect of the invention there is provided a method of preparing an array of the invention, comprising (a) obtaining a structure as described above, and (b) contacting at least one profiled feature with a biomolecule.

In yet a further aspect of the invention there is provided an assay method comprising the steps of:

(i) contacting an array described above with a test sample that may contain an analyte that binds to the at least one biomolecule adsorbed on the surface within the at least one profiled feature;

(ii) detecting binding of the analyte and the adsorbed biomolecule.

### DETAILED DESCRIPTION OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference to any prior art in this specification is not, and should not be taken as, an acknowledgment or any form of suggestion that that prior art forms part of the common general knowledge in Australia.

The term “molecule-adsorbing” as used herein refers to materials and surfaces capable of binding molecules. The molecules may be bound to the surface or material by any interaction which is capable of maintaining the molecule in contact with the surface or material. For example, the surfaces or materials may bind molecules by ionic interactions, electrostatic forces, hydrogen-bonding or hydrophobic interactions. Alternatively, the surfaces or materials may bind molecules by the formation of covalent bonds.

The structures of the present invention are preferably micro-structures for use in microarrays and micro-assays. As used herein the term “micro” means small. A micro-structure may range in width from a few microns to millimeters and may contain thousands of profiled features, each profiled feature having a width or diameter in the range of submicrons to 10 s of jams.

The first layer comprising a substantially non-ablatable material may be any material capable of adsorbing molecules. Preferably the first layer is substantially transparent to laser wavelength. Preferably the first layer is polymeric and preferably the polymer is capable of thermal degradation under laser ablation conditions to provide a surface having diverse surface properties. Especially preferred are polymers that thermally degrade to provide a surface having localized areas which are hydrophobic, hydrophilic, acidic, basic, charged or neutral. Suitable polymeric materials include polyacrylates, polycarbonates, polystyrenes, fluorine-containing polymers, polyethylenes and their derivatives. Examples of suitable polymers include polymethylmethacrylate (PMMA), polyacrylic acid, polyacrylonitrile, polymethacrylate, styrene-acrylonitrile copolymers, butadiene-styrene copolymers, polyalkylstyrenes for example polymethylstyrene, polyethylstyrene and polypropylstyrene, and polytetrafluoroethylene (PTFE). Particularly preferred is PMMA.

The thickness of the first layer is in the range of fractions of microns to tens of microns. Preferably the thickness of the first layer is about 1 micron.

In a preferred embodiment, the surface of the first layer that is exposed has at least two and preferably a plurality, of localized areas having molecule-adsorbing capacities for molecules with different adsorbing properties. For example, each localized area may present a hydrophobic, hydrophilic, acidic, basic, charged or neutral surface and therefore has the capacity to adsorb molecules which have a surface that is complementary or attracted to the localized area of the surface. A localized area of hydrophobicity will adsorb molecules that also have a hydrophobic surface, whereas a localized area which has a negatively charged surface will adsorb molecules having a positively charged surface. These localized areas having molecule-adsorbing capacities for molecules with different adsorbing properties may occur in the same profiled feature or in different profiled features. The term “molecule-adsorbing capacity” refers to the surface properties presented by a localized area.

The localized areas may be formed in a structured or unstructured manner. For example, a structured surface where the localized areas form a predetermined pattern on the surface first layer may be formed. Such a pattern may be, for example, alternating localized areas of hydrophobic and hydrophilic surfaces and surfaces with different chemistries (eg: NH₂, CO₂H, OH).

Alternatively, the surface may be unstructured where the laser ablation method used results in areas having different molecule-adsorbing capacities. For example, areas of hydrophobicity and hydrophilicity may be obtained depending on the amount of thermal energy to which a particular area is exposed. For example, and without being bound by theory, based on AFM topography and lateral force imaging, as well as the knowledge regarding laser ablation, the following mechanism of formation of the observed structures and subsequent variations in molecule adsorption may be proposed. Laser exposure (ns) causes the overheating of a polymer to a point where the polymer is melted and chemical reactions start to occur. The expected reactions would be, in the order of increasing pyrolysis temperature, (i) the termination of the side ester groups at one of the C—O bonds, resulting in a more hydrophilic material; (ii) depolymerization of the main chain, preserving the same hydrophobicity; and if the process is quick enough (iii) the breaking of the side bonds, resulting in a more hydrophobic material. Therefore, we can hypothesize that there are three regions in the micro-well. At the center of the ablated line where the thermal energy would reach a maximum the decomposition is the most advanced, the polymer would experience the breaking side chain C—C groups, and possibly condensation reactions leading to aromatic rings, resulting in a more hydrophobic material. Between the center and the edge of the ablated line the polymer undergoes depolymerization only. At the edges of the ablated line, where the thermal energy has the lowest levels and the remaining metal layer absorbs the overheating, the polymer is de-esterified (with generation of gases), melted and expulsed over the edges of the micro-well, resulting in a porous, more hydrophilic zone. Therefore an unstructured surface in a profiled feature may have a central hydrophobic area and a hydrophilic area at its edges.

Preferably the surface of the first layer that is exposed by laser ablation is textured rather than flat: The surface preferably contains pores and valleys within a profiled feature. FIGS. 10a and 10b show AFM topographical and lateral force images respectively, of a micro-channel fabricated via ablation of a gold layer disposed on a PMMA layer. FIG. 11a is a three dimensional representation showing the rugosity of the surface inside of the micro-channel, as compared with the surface outside the channel (FIG. 11b). FIG. 10c shows the topography of the channel (thin line) as having a shoulder at each edge of the micro-channel and two valleys in the centre. The hydrophobicity of the regions of the micro-channel (thick line) shows the central region is a hydrophobic region and towards the edges of the micro-channel, hydrophobicity decreases to provide a more hydrophilic region.

The second layer comprising an ablatable material may be any material that is opaque to laser wavelength and is able to be evaporated under ablation conditions. Preferably the ablatable material is a metal that can be deposited in a thin layer. Suitable metals include Au, Cr, Ag, Mg, Ti, V, Mn, Fe, Co, Ni, Cu, Zn, Cd, Pt, Pd, Rh, Ru, Mo, W and Pb. Particularly suitable ablatable materials include Ag, Cr, and Au. Particularly preferred is Au.

The thickness of the second layer is in the range of tens of nanometers. Preferably the thickness of the second layer is in the range of 20-60 nm, more preferably about 30 nm.

Laser ablation of at least a portion of the second layer exposes the top surface of the first layer and may cause thermal degradation of the localized areas of the exposed surface of the first layer. The laser ablation of the second layer forms shallow profiled features in the structure. The shallow profiled features may be any shape but are preferably square or rectangular and may be in the form of a well or a channel. The bottom of the profiled feature is formed by the top surface of the first layer. The depth of the shallow profiled feature corresponds to the thickness of the ablatable layer and is preferably less than 100 nm, more preferably 50 nm or less.

Preferably the structure comprises a further blocking layer. The blocking layer may be any material capable of preventing binding of molecules. The blocking layer is ablated together with the second layer. However, the blocking layer remaining on top of the second layer after ablation repels the molecule to be adsorbed on the surface of the first layer or the analyte or recognition molecule from the non-ablated portions of the second layer. The blocking layer may include any polymer or protein that is unreactive and will not interact with the molecule to be adsorbed or their complementary components. Suitable blocking materials include inert polymers such as polyethylene glycol and polyethylene oxide and inert proteins such as bovine serum albumin (BSA).

Alternatively, the blocking layer may be a Self Assembled Monolayer (SAM), formed from for example, alkanethiols, alkylsiloxanes and fatty acids. SAMs formed from alkanethiols, such as C₆-C₂₀alkanethiols, are preferred for use when the second layer is formed from Au or Ag. Preferably the terminal end of the alkane group of the alkanethiol is a methyl group or is substituted with a functional group such as a carboxylic acid, amino group or a hydroxy group. Particularly useful are mixtures of alkanethiols having variable terminal substitution, for example a SAM assembled from a mixture of alkanethiols where the alkane terminus is methyl or substituted with carboxylic acid, amino or hydroxy groups. If the properties of the molecule to be adsorbed are known, the blocking layer can be tailored to repel the molecule. For example, if the molecule to be adsorbed has a number of negative charges, then the SAM may be assembled from alkanethiols that also present negative charges thereby repelling the molecule from the surface of the blocking layer.

A further alternative is the use of multilayer thin films prepared by sequential assembly of nanocomposite materials such as polyelectrolytes. Such multilayer thin films can be prepared to present an inert surface on top of the second layer. In a similar manner to SAMs, the polyelectrolyte molecules may have functionality which will repel the molecule to be adsorbed from the surface of the blocking layer. The use of multilayer thin films of electrolytes to present inert surfaces is known in the art.

Suitably the thickness of the blocking layer is in the range of a few nanometers for inert polymer blocking layers and SAM blocking layers to tens of nanometers for inert proteins.

In a preferred embodiment, the structure includes an orderly arrangement of a plurality of profiled features. The plurality of profiled features may form a plurality of wells or a plurality of channels. In a particularly preferred embodiment, the plurality of profiled features may be arranged in a pattern that is capable of identifying a feature of an array formed from the structure. For example, a plurality of channels may be formed in a “bar code” type arrangement and each structure may contain a plurality of different bar code type arrangements. Each bar code type arrangement may be used to encode particular information about an array prepared from the structure or the samples applied to the array in an assay. The term “informationally-addressable” as used herein refers to the ability of the profiled features to encode information about an array or an assay.

In one embodiment, each informationally-addressable profiled feature or bar code may be used to identify a different molecule adsorbed on the surface of the first layer in a profiled feature of an array or may be used to identify a series of different concentrations of a single molecule adsorbed on a respective series of bar code type arrangements. Alternatively, each bar code arrangement may be used to encode information about an assay in which the array is to be used. For example, the bar code may be used to identify the source of the analyte or recognition component. In a diagnostic assay where each profiled feature forms a bar code and each profiled feature has the same molecule, eg. a protein or gene, adsorbed on the exposed surface of the first layer, the bar code could be used to identify the patient who is being tested.

The structure of the invention preferably further comprises a substrate that supports the bilayer comprising the first and second layers. The substrate can be made of any material suitable for supporting the first and second layers and which is capable of withstanding the conditions used in preparing and using the structure. Examples of suitable substrates include quartz glass, mesoporous silica, nanoporous alumina, ceramic plates, glass, graphite and mica. Preferably the substrate is ordinary glass. Alternatively, the substrate may be part of the apparatus used for fabricating the structure or array, or for performing the assay. The structure may be prepared on the surface of a substrate and then removed or transferred to another substrate.

The structure of the invention may be used in the preparation of an array, where at least one profiled feature has a molecule adsorbed on the exposed surface of the first layer. Preferably the structure has a plurality of profiled features and each profiled feature contains a molecule adsorbed on the exposed surface of the first layer. Each profiled feature may contain the same or a different molecule.

The molecule adsorbed on the molecule-adsorbing surface of the profiled feature may be any molecule of interest. For example, the molecule may be a biomolecule such as a gene, DNA, RNA, oligonucleotide, protein, polypeptide, peptide, polysaccharide, oligosaccharide, antibody, antigen, enzyme, enzyme substrate or enzyme inhibitor. Alternatively, the molecule could be a drug or potential drug derived from natural or synthetic sources.

The present invention is particularly useful for enabling the adsorption of proteins. Proteins present extremely varied molecular surfaces, for example, hydrophilic, hydrophobic, acidic, basic, neutral or charged surfaces, and may be sensitive to denaturation upon adsorption on a surface. The variation in the molecule-adsorbing capacity in the surface of the first layer provides localized areas that may interact with different proteins having different surface properties or may interact with the same protein by a different surface. This latter interaction will ensure that at least some of the adsorbed protein will have a recognition site, such as an active site, receptor or binding site, exposed for use in an assay. The latter interaction may result in an increase in the amount of protein that is bound to the exposed surface compared to a surface that lacks variation in the molecule-adsorbing capacity. As can be seen in FIG. 9, large proteins, for example, human serum albumin (HSA) and immunoglobulin (IgG) may bind to a surface having localized areas with different molecule-adsorbing capacities at about three times the level found with an unvaried flat surface. Smaller proteins, such as lysozyme, myoglobin and α-chymotrypsin may bind to the surface having localized areas of molecule-adsorbing capacity at about 10-12 times the level found on an unvaried flat surface.

If the localized areas having different molecule-adsorbing capacities are arranged in a predetermined pattern, for example, hydrophilic areas at the edges of a channel or well and hydrophobic areas in the centre of a channel or well, alignment of the adsorbed biomolecules may occur. This may result in increases in the number of biomolecule-analyte interactions that occur during an assay. The immobilized proteins may be used to probe protein-protein, enzyme-substrate, protein-DNA, protein-oligosaccharide or protein-drug interactions.

The arrays of the invention may be used in assays to probe interactions between an adsorbed molecule and an analyte or recognition component. Such interactions include RNA/DNA-RNA/DNA, RNA/DNA-protein, RNA/DNA-drug, RNA/DNA-oligosaccharide, protein-protein, enzyme-substrate, enzyme-inhibitor, antibody-antigen, protein-RNA/DNA, protein-oligosaccharide, oligosaccharide-protein, oligosaccharide-oligosacchride, oligosacchride-drug or drug-drug interactions. For example, an assay may be used to find substrates or inhibitors of a particular enzyme adsorbed on the exposed surface of the first layer, or may be used to determine a mechanism, such as whether the enzyme inhibitor is competitive or non-competitive. The arrays may also be used to explore the interaction of the biomolecules with the surfaces. For example, the effects of such interactions on bioactivity of the biomolecules or whether the interactions cause denaturation of the biomolecules.

The assay method of the invention may be performed by contacting the adsorbed molecule(s) in an array with a test sample, the test sample potentially containing an analyte or recognition component that will bind to the adsorbed molecule. The presence or absence of the analyte or recognition component in the test sample can then be detected. The term “analyte” or “recognition component” as used herein refers to a molecule that is recognised by and interacts with the molecule adsorbed in the profiled feature.

The adsorbed molecule and analyte or recognition component may be selected from pairs of complementary compounds such as a single strand of DNA, RNA or an oligonucleotide and their complementary strand, an antibody and an antigen, an enzyme and a substrate or an inhibitor, a drug and a receptor.

The coupling of the adsorbed molecule and the complementary component may be detected by any detection means known in the art. For example, fluorescence detection may be used, where a fluorescent marker is tagged onto the adsorbed molecule or the analyte or recognition component or may be bound to the adsorbed molecule/analyte or recognition component pair in a further step. Preferably the marker is tagged onto the analyte or recognition component or may be bound to the adsorbed molecule/analyte or recognition component pair in a further step. Other suitable means of detection includes the use of luminescent, phosphorescent or radioactive markers or the use of nanoparticles or magnetic beads as known in the art.

The assay may be used as a diagnostic assay or may be used in high throughout screening of molecules. For example, in a diagnostic assay the array containing many different DNA molecules indicative of specific genes may be prepared and a test sample from a patient, for example, serum, added to the array by “flooding” the array and then after appropriate washing and the addition of a detection marker, the coupling of complementary DNA sequences can be detected. This may give an indication of whether the patient has a specific gene or a mutation in a specific gene.

Alternatively, an array of profiled features all containing the same antigen or antibody, could be prepared. Test samples obtained from different patients suspected of having a particular disease caused by the antigen, could be added, one test sample to each of the profiled features. After washing and addition of an appropriate marker, the coupling of antigen and antibody can be detected. The samples in which an interaction between antigen and antibody are detected, can be used to indicate the presence of the disease state in the patients providing the samples.

The present invention may be adapted for use in known genetic and protein assays. Preferably the assays are protein assays such as antibody assays.

In another embodiment the assay may be used in high throughput screening. For example, an array containing a variety of drug targets which are known to be involved in the initiation or progress of a disease state, can be prepared. The array can be contacted with a potential drug and its interaction with the drug targets assessed.

The structures and arrays may be prepared by obtaining a substrate supporting a first layer comprising a molecule-adsorbing, substantially non-ablatable material and a second layer comprising an ablatable material disposed on the first layer. The substrate may be coated with the first layer by any suitable technique, for example, sputter coating, spin coating. Preferably the first layer is coated on the substrate by spin coating.

Similarly the second layer may be applied to the first layer by any suitable means. For example, sputter coating, spin coating or electroplating. Preferably the second layer is applied by sputter coating.

1. Optionally the surface of the second layer is blocked by the application of a blocking layer. For example, the blocking layer may be an inert polymer, such as polyethylene oxide or polyethylene glycol, or an inert protein, such as BSA. A protein blocking layer may be applied by immersion and incubation of the substrate supporting first and second layers in a solution of blocking protein (1-5% w/v BSA in an appropriate buffer) at room temperature for 15 minutes to 1 hour or by soaking in the protein solution or via addition of a droplet of a protein solution. A SAM blocking layer may be prepared by immersing the substrate supporting the first and second layers in a solution of compound that will form the SAM in an appropriate solvent, for example, a 1-2 mM solution of decanethiol in ethanol. Other methods of forming SAMs are known in the art. A multilayer thin film is formed by the steps of immersion of the substrate supporting the first and second layers in a solution of first polyelectrolyte having a first charge and then immersion of the substrate obtained from the first immersion step in a solution of second polyelectrolyte having a charge complementary to the first polyelectrolyte. Adjusting the pH of the solutions containing the electrolytes results in differences in the structure of the layers and may provide either sheet like layers or disordered layers. A polymeric blocking layer may be applied by spin coating.

The second layer is then subjected to laser ablation such that at least a portion, and preferably a plurality of portions, of the second layer is ablated to expose the surface of the first layer.

The fabrication of structures and arrays according to at least one embodiment of the invention are shown in FIG. 1 and 2. In FIG. 1, a substrate (1) coated with a first layer comprising a molecule adsorbing, substantially non-ablatable material (2), a second layer comprising an ablatable material (3) and a blocking layer (4), is subjected to laser ablation to produce a profiled feature (5) to which a droplet of molecule (6) to be adsorbed is added with a picoliter pipette (7). In FIG. 2, an array of different biomolecules is prepared.

The laser wavelength used for ablation may be between 100 nm to 1200 nm, preferably 150 nm to 1100 nm. A typical high energy wavelength is in the range of 150 to 300 nm. A typical low energy wavelength is in the range of 300 nm to 1100 nm.

The laser ablation process may be performed at atmospheric pressure or below atmospheric pressure. Preferably, ablation is performed at below atmospheric pressure to assist in the removal of debris.

Preferably the fabrication platform consists of a computer controlled laser ablation system, comprising a research-grade inverted optical microscope, a pulsed nitrogen laser emitting at 337 nm, a programmable XYZ stage and a Pico-litre pipette mounted on the XYZ stage. Preferably the profiled features formed during the laser ablation step are micro-wells or micro-channels.

Wells having diameters of from sub-micron widths to about 50 μm are able to be prepared and are useful in preparing arrays. Wells having diameters of 5-20 μm, 1-5 μm and submicron widths are readily achieved by focussing through a 20× dry objective, a 40× dry objective or a 100× oil immersion lens, respectively. Preferably the wells have widths in the range of 5 μm-50 μm, more preferably 5 μm to 10 μm.

Channels having from submicron widths to about 50 μm widths may also be prepared. The channel may be any length but is preferably 5 to 200 μm long. In a similar manner to wells, channels having diameters of 5-20 μm, 1-5 μm and submicron widths are readily achieved by focussing through a 20× dry objective, a 40× dry objective or a 100× oil immersion lens, respectively. Preferably the channels have widths in the range of 5-50 μm, more preferably 5-10 μm.

In a preferred embodiment the XYZ stage is programmed to allow the laser ablation of vertical lines forming channels at different distances to form a pattern that is informationally addressable.

A structured surface may be prepared by exposing a number of localised areas to laser exposure with different exposure times, different powers, and/or different wavelengths, all translating in different energies absorbed by the ablatable material. Additionally, the thickness of the ablatable material may be varied, therefor requiring different energies of ablation. A first localised area may be exposed to a high energy wavelength for a short period of time, in the order of femtoseconds, resulting in ablation of the ablatable material but minimal build up of thermal energy and therefore minimal decomposition of the surface of the molecule-adsorbing, non-ablatable material. A second adjacent localised area may be exposed to a lower energy for a longer period of time, for example in the order of nanoseconds to microseconds, resulting in a greater build up of thermal energy and therefore greater thermal decomposition of the surface of the molecule-adsorbing, non-ablatable material occurs. The process may be repeated to provide a number of localised areas having different or alternating adsorbing properties on one surface.

The array of the present invention may be prepared by adsorbing molecules of interest onto the exposed surface of the first layer within the profiled feature(s). One method of fabricating the array is to laser ablate a plurality of portions of second layer to form a plurality of profiled features and then to “flood” the structure with a solution containing the molecule to be adsorbed. This method may provide an array having the same molecule adsorbed in each profiled feature if application of the solution of molecule to be adsorbed occurs after the laser ablation process is complete and all profiled features have been fabricated. Alternatively, a portion of the structure may be ablated to provide a portion of the profiled features of the array, the surface may then be flooded with a molecule to be adsorbed. This two step process may be repeated multiple times to build up the entire array. Each two step process may use the same or different molecule in the adsorption step. Preferably each two step process uses a different molecule in the adsorption step to provide an array having a plurality of profiled features with at least two different molecules adsorbed on their surfaces. Another method of fabricating the array is to laser ablate a plurality of portions of the second layer to form a plurality of profiled features in a “spatially-addressable” mode and then deposit the molecule to be adsorbed in each of the profiled features with a Pico-liter pipette. This technique may be used to achieve an array having a different molecule adsorbed in each profiled feature or at least some profiled features having different molecules adsorbed in them. As used herein the term “spatially-addressable” refers to the ability to apply a solution to an individual profiled feature.

In a similar manner when performing the assay, the test solution of analyte or recognition component may be applied to an array containing different molecules in at least some of the plurality of profiled features, by flooding the array with the test solution containing the analyte or recognition component. Alternatively, different test solutions containing different analytes or recognition components may be applied to each profiled feature using the spatially addressable mode described above.

### BRIEF DESCRIPTION OF THE FIGURES

FIG. 1 schematically represents the procedure for preparing structures and arrays by laser ablation.

FIG. 2 schematically represents a procedure for fabrication of an ablated array, with fluorescent images before (middle top and middle bottom) and after antibody deposition (right). The ablated micro-wells are 100×100 μm.

FIG. 3 represents fluorescence images of anti-chicken IgG AlexaFluor 546-conjugate deposited in profiled features prepared by laser ablation of Au deposited on PMMA. The profiled areas were prepared using different laser doses. Upper left area ablated with 60% laser power, bottom left—100%, upper right—40% and bottom right—80%.

FIG. 4 represents topographical (left) and friction force (right) images of a Au-PMMA bilayer struction exposed to different laser doses. Upper left area ablated with 40% laser power, bottom left—60%, upper right—80% and bottom right—100%.

FIG. 5 represents fluorescence images of anti-chicken IgG AlexaFluor 546-conjugated deposited on the ‘bar code’ micro-structure fabricated in a Au-PMMA bilayer. From the left: 1^{st} line, ablation with 100% of laser power at a rate of 20 pulse/s, and the writing speed of 10 μm/s; 2^{nd} line, ablation with 100% of laser power at a rate of 20 pulse/s, and a writing speed of speed 10 μm/s, repeated twice; 3^{rd} line, ablation with 100% of laser power at a rate of 20 pulse/s, and a writing speed of 20 μm/s. The inset represents a pseudo-map of the intensity of the fluorescence.

FIG. 6 represents topographical (top left) and friction force (top right) images of a channel created by the laser beam (100% laser power, 20 pulse/s and 10 μm/sec writing speed). The bottom plot represents the profile of a transversal section of the channel.

FIG. 7 represents fluorescence images of labelled protein adsorbed on structures fabricated via laser ablation at different power levels (conditions as in FIG. 6). The amplification of fluorescence (in inset on each line) and the pseudo-map of the intensity of the fluorescence (inset upper left) compared with the hydrophobicity map (inset upper right) reveal a ‘fine structure’ of protein deposition, preferentially on the hydrophilic edges of the channel and on the hydrophobic ridge on the center of the channel.

FIG. 8 shows detection of specific antigens in high density ‘bar code’ array format demonstrated by incubation of the array with fluorescently labelled individual or collective antibodies. On the top—a fragment of ‘bar code’ array of two different proteins in the bright field; on the bottom—fluorescent image of the same array with specific recognition by anti chicken IgG AlexaFluor 546 conjugate.

FIG. 9 is a graphical representation showing modulation of protein adsorption in micro/nano-channels having localised areas of molecule-adsorbing capacity.

FIG. 10a is an AFM topographical image of a channel fabricated via laser ablation of a 30 nm Au layer on top of P

FIG. 10b is an lateral force image of a channel fabricated via laser ablation of a 30 nm Au layer on top of PMMA.

FIG. 10c is a graphical representation of the topography and hydrophobicity of a profiled feature. The topography (thin line) shows a cross section of a channel having a shoulder at each edge and two valleys in the central region (I). The hydrophobicity (thick line) shows a central hydrophobic region and a reduction in hydrophobicity towards the sides of the channel.

FIG. 11a is a three dimensional representation showing the rugosity of the surface of the first layer in a profiled feature.

FIG. 11b is a three dimensional representation showing the rugosity of the surface of the second layer in an unablated area of the structure.

FIG. 12 shows protein adsorption on PMMA microstructures. The first row presents bright field images. The fluorescent images relate to different protein concentration in solution as follows: 0.014 mg/ml (second from top); 0.07 mg/ml (third from top); 0.14mg/ml (bottom).

FIG. 13 shows AFM topographical images (tapping mode) of the adsorbed α-chymotrypsin (top) and IgG (bottom) on micro-channels.

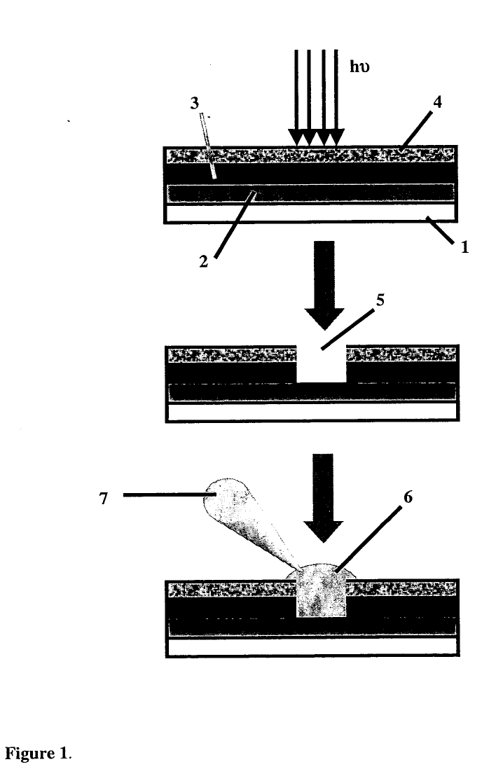

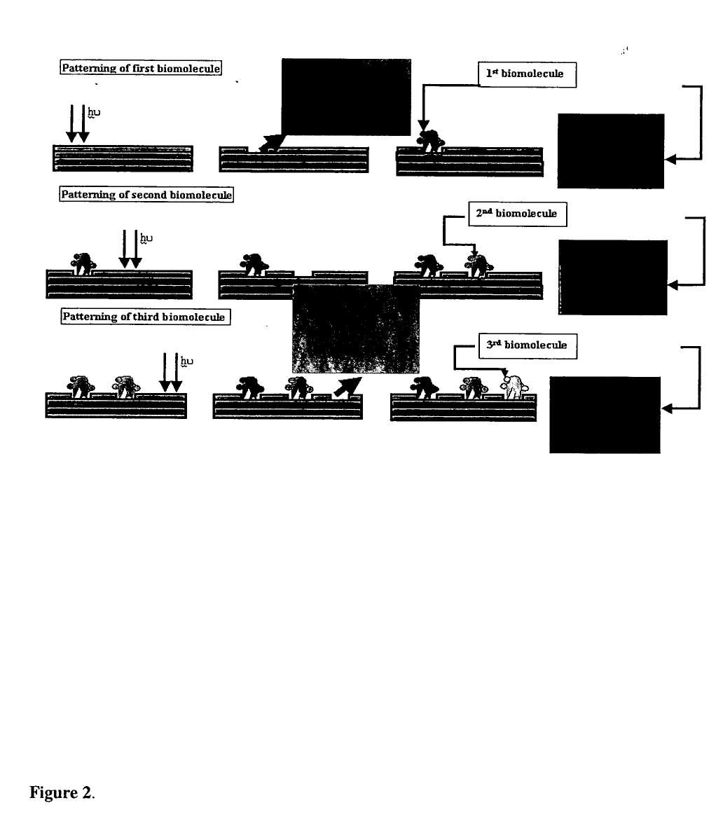

### EXAMPLES

Atomic Force Microscopy (AFM) can be used not only for fine mapping of the topography of a surface, but also for probing the physics and chemistry of the surface. In this context, AFM has been used to probe intermolecular interactions with pN sensitivity and spatial resolution of nanometers, Noy et al. (1997). When imaging under ambient conditions, the capillary condensation between the tip and sample surfaces reflects the relative degree of hydrophilicity and can be used as a basis for discriminating between hydrophobic and hydrophilic groups, Wilbur et al. (1995). The image contrast in a lateral force map is effectively a measure of tip-to-surface friction. Frictional force follows the generalized Amonton's law, Noy et al. (1997), Wilbur et al. (1995), Sinniah et al. (1996), Vezenov et al. (1997):

F_{f}=μF_{N}+Fₒ (1)

Where μ is friction coefficient, F_{N} is the lever-induced normal force and Fₒ is ‘residual force’ which correlates with adhesion force between the tip and the sample surfaces.

Previous studies, Noy et al. (1997), have shown that the interaction forces between tips and samples which both terminate with hydrophobic groups are small. Observed interaction forces are also small when one of the surfaces terminates with hydrophobic groups and the other terminates with polar groups, whereas significant interactions are observed when both the tip and sample surfaces terminate with hydrophilic groups (hydrogen-bonding).

The SiN₄ tip used in the present study is hydrophilic due to the native oxide surface layer. The frictional force is therefore higher as the tip is scanned across a hydrophilic surface, compared to a hydrophobic surface.

Protein Preparations: Several immunoglobulins (IgG's), i.e. bovine IgG, chicken IgG, human IgG, rabbit IgG, and affinity isolated antigen-specific corresponding antibodies (whole molecule) were purchased from Sigma. Streptavidin and fibrinogen were used as control proteins. The IgGs were prepared as stock solutions at a concentration of 2 mg/ml and diluted with TBS to 100 μg/ml as working solutions prior to experiments.

Three fluorescent labels have been used, namely: fluorescein isothioiocyanate (FITC), AlexaFluor 456, and AlexaFluor 350. The FITC and AlexaFluor fluorescent tags have been conjugated to the selected proteins using FluoroTag Kits purchased from Sigma and Molecular Probes, respectively. The labelling procedure was carried out according to the instructions of the manufacturer. Each protein was used in concentration of 2 mg/ml. The labelled proteins were purified from unconjugated fluorescent dyes using a Sephadex G-25 column. The concentration of antigen conjugate was determined by UV-Vis spectroscopy. The Fluorescent dye/Protein molar ratio of the purified protein was determined by measuring the absorbance at 280 nm (for protein), and 495 nm (for FITC), 556 nm (for AlexaFluor 546), and 346 nm (for AlexaFluor 350).

Preparation of the micro-fabricated structures. Glass slides or cover slips (0.17 mm thick, 24×24 mm, Knittel) were sonicated in Nanopure water for 30 min and washed copiously with filtered (0.2 μm) Nanopure water (18.2 MΩ/cm), dried under a stream of high purity nitrogen, and then primed with hexamethyldisilazane. A 4 wt % solution of PMMA in propylene glycol methyl ether acetate (PGMEA) 99% (purchased from Sigma Aldrich Co.) was spin-coated at 3000 rpm for 40 s using a Specialty Coating Systems spin coater (Model P6708). For these conditions, the PMMA film thickness was 0.5 μm. The coated substrates were then soft baked at 85° C. for 30 min, and stored in a desiccator prior to and after gold deposition. The deposition of gold was done using a sputtering SEM-coating unit E5100 (Polaron Equipment Ltd) at 25 mA for 90 s at 0.1 Torr. For these conditions, the gold film thickness was 50 nm. The gold-layered substrata were then incubated with bovine serum albumin (BSA) by immersion in a 1% w/v BSA 10 mM phosphate-buffered saline (PBS) solution (pH 7.4) at room temperature for approximately 1 h, and then rinsed with PBS followed by Nanopure water.

The laser-based microfabrication of gold-coated polymeric films can be readily accomplished with commercially available microscope adaptations. The system (Cell Robotics, Inc.) comprised a Nikon Eclipse TE300 inverted microscope, coupled with a computer-controlled, pulsed nitrogen laser emitting at 337 nm with a maximum intensity of 120 μJ/pulse and focused directly through the microscope objective lens.

Quantification of surface-related processes. The hydrophobicity of the films was estimated by contact angle measurements. Advancing contact angles were measured on sessile drops (2 μl) of Nanopure water at room temperature (20-23° C.) in air using a contact angle meter constructed from an XY stage fitted with a (20 μl) micro syringe, a 20× magnification microscope (ISCO-OPTIC, Germany) and a fibre-optic illuminator. The observed values were averaged over six different readings.

Atomic Force Microscopy (AFM) was carried out on an Explorer system (ThermoMicroscopes) in the normal contact mode. The AFM system is based on detection of tip-to-surface forces through monitoring optical deflection of a laser beam incident on a force-sensing/imposing lever. Several scanners were used in order to cover the scales of lateral topographical and chemical differentiation; the fields-of-view ranged from 100×100 μm down to 8×8 μm. The analyses were carried out under air-ambient conditions (temperature of 23° C. and 45% relative humidity). Pyramidal-tipped, silicon nitride cantilevers with a spring constant of 0.032 N/m were used. As the tip is scanned across the surface, the lateral force acting on the tip manifests itself through a torsional deformation of the lever, which is sensed by the difference signal on the Left-Right signal on the quadrant detector. The difference signal can be plotted as a function of XY location in the topographical field of view, and the resulted friction force image can then be correlated directly with the topographical image.

The attachment of fluorescently labelled proteins on the ablated micro-structures was visualized using and analysed using two different microscopic systems. The first is a Nikon TE300 inverted microscope, coupled with an epi-fluorescence illumination unit fitted with filter sets specific towards FITC (CR101, Chroma Technology) and AlexaFluor (XF108-2, Omega Optical, Inc.). The second was a Nikon Microphot FX microscope with a UV light source (Nikon Mercury Lamp, HBO-100 W/2; Nikon C.SHG1 super high pressure mercury lamp power supply) at 100× objective. These images were captured on a Nikon camera (FX-35WA). The fluorescent images were observed using an intensified CCD video camera, Lumi Imager (Photonic Science), and processed using PaintShop Pro (Jasc Software). The fluorescence intensities were analysed using Gel-Pro Analyser software, version 4.0.

Multi-analyte antibody assay. The assays fabricated as described above comprised different IgGs (1-7 μl of 100 μg/ml), either fluorescently labelled for the visualization of the selectivity of protein attachment, or unlabelled for the visualization of the selectivity of protein recognition by labelled antigens, deposited onto micropatterned ablated areas as described above.

For assay fabrication and process monitoring and optimization, IgG conjugates with FITC and AlexaFluor's (2-7 μl of 100 μg/ml) were deposited onto fabricated ablated geometries either in a ‘blanket’ mode, flooding the whole surface of the assay; or in a spatially-addressable manner, using the pico-liter pipette. For the ‘blanket’ deposition, the slide was incubated for 30 min at room temperature in a humid chamber, then the slide was washed three times with PBS and twice with Nanopure water. The spatially-addressable deposition used a pico-liter pipette (CellSelector module, Cell Robotics Inc.) mounted on the same precision XY stage. Very small amounts, usually around few hundreds nanoliters down to hundreds of picoliters, can be deposited in precise locations, usually within micron-range precision.

For the testing of the assays, the IgGs-covered surfaces were incubated individually or collectively with corresponding fluorescently labelled antibodies and control proteins (e.g. fibrinogen, streptavidin) for 2 h at room temperature. The assay structures were then washed three times with PBS, and twice with Nanopure water. The images of the selectively recognized patterned features were analysed as described above.

### Example 1

### Assays in Array Format

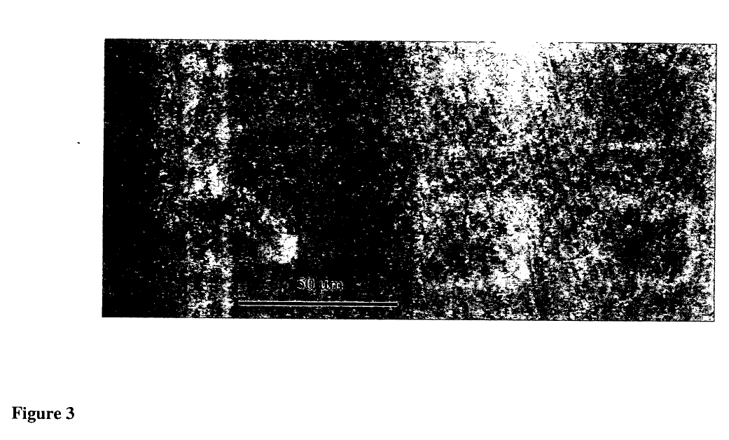

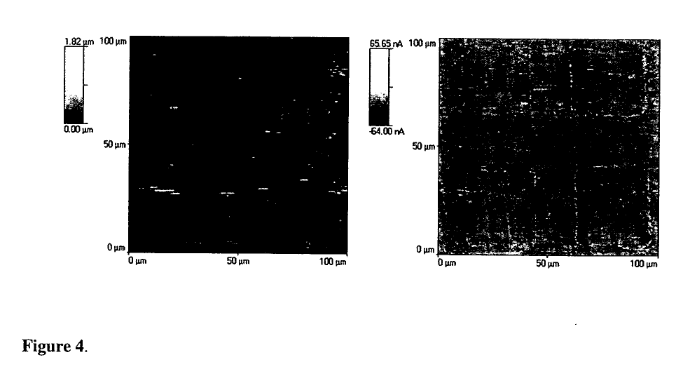

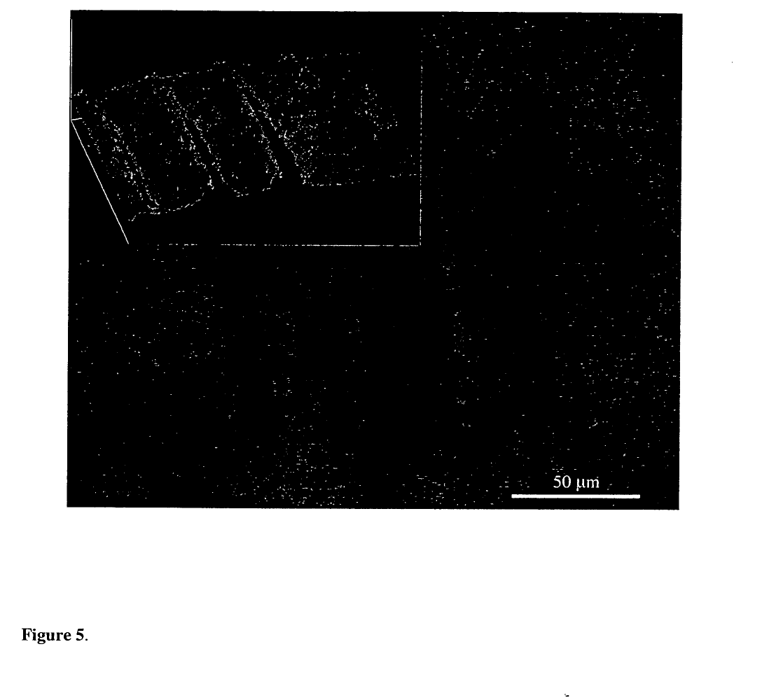

In order to explore the interaction between laser power—surface properties—molecule adsorption, and in particular, protein adsorption, 50×50 μm areas were ablated at different ablation energies. A few lines were also ablated to form channels. Fluorescently labelled antibody (anti-chicken IgG AlexaFluor 546) was deposited and incubated. The results are shown in FIG. 3. It appears that the proteins deposit primarily on the regions at the edges of the ablated areas and that, after a certain power threshold (around 50%), this concentration levels off. In principle, the higher concentration of the protein could be an artifact resulting from the verticality of the wall (apparently thicker protein layer seen from the top of a vertical wall). However, the height of the wall is not large (around 50 nm) and, more importantly, the AFM analysis (FIG. 4) points out the real differences in the material characteristics near the edges of the ablated area. First, the lateral force measurements, taken before protein deposition, proves that the outer surface (Au) has a similar hydrophobicity with the inner area (PMMA) in line with the similar contact angles for these two materials (around 65° and 70°, respectively). Second, the AFM-measured topography shows that indeed the bottom of the well is deeper and rather flat, except for the edges that are elevated above the level of the gold layer. Third, the AFM-mapping of the lateral force clearly shows a hydrophilic rim at the edges of the ablated area (areas in FIG. 5, right side), possibly guarded by thinner hydrophobic stripe (brighter and darker areas in FIG. 5 right, respectively). Moreover, the width of the rim seems to be rather independent of the laser power.

### Example 2

### Bar Code Assays.

Linear structures which both decrease the actual amount of protein used for deposition, especially if a spatially-addressable deposition is used, as well as increase the capacity for miniaturization in a lateral if not in a 2D manner, were fabricated. Another benefit of this approach arises from the possibility to encode the information (e.g. type of antibody, concentration) through a combination of vertical lines in a ‘bar code’, ‘informationally-addressable’ mode and not in a 2D, spatially-addressable mode like in the classical arrays. The results also demonstrate, inter alia, the complexities of protein adsorption in fabricated channels, with the resolution of the variation of the protein concentration in the nanometer range. These complexities are likely to have an increasingly important impact in microfluidics, especially for devices that comprise nano-channels.

Proteins adsorb either via hydrophobic interactions between hydrophobic patches on the molecular surface and adsorbing surfaces, or via weaker electrostatic interactions between charged patches and charged surfaces. It follows that the protein will be adsorbed at the center of the well and on the porous zone at the edges. On rectangular ablated areas, where the center of the well is ablated by subsequent sweeps of the laser beam, much of the protein adhesion occurs at the edges of the ablated area.

The processing conditions (e.g. laser power, speed of writing) were tested in order to clarify the optimal surface treatment that will facilitate the best and reproducible protein attachment. The results of this experiment are presented on FIG. 5. Protein attachment reached a maximum on the lines ablated with 100% of the laser power and at the highest pulse rate (i.e. 20 pulse/s, line no. 3 from the left in FIG. 5). When using the same total energy, but via the ablation with a rate of 10 pulse/s repeated twice (lines no. 1 and 2 from the left, respectively) the protein adsorption was less apparent. Therefore further experiments used this optimized parameters for the fabrication of the protein patterns.

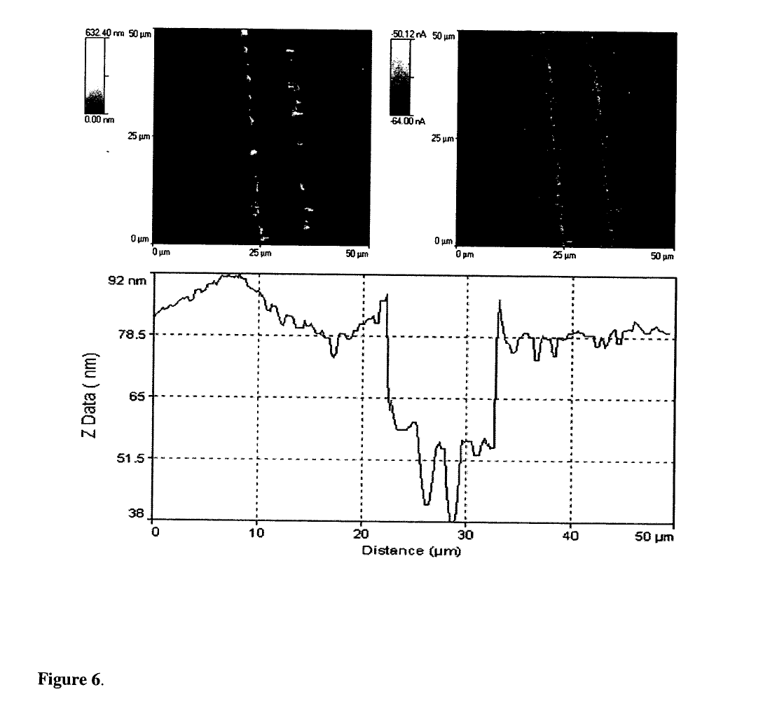

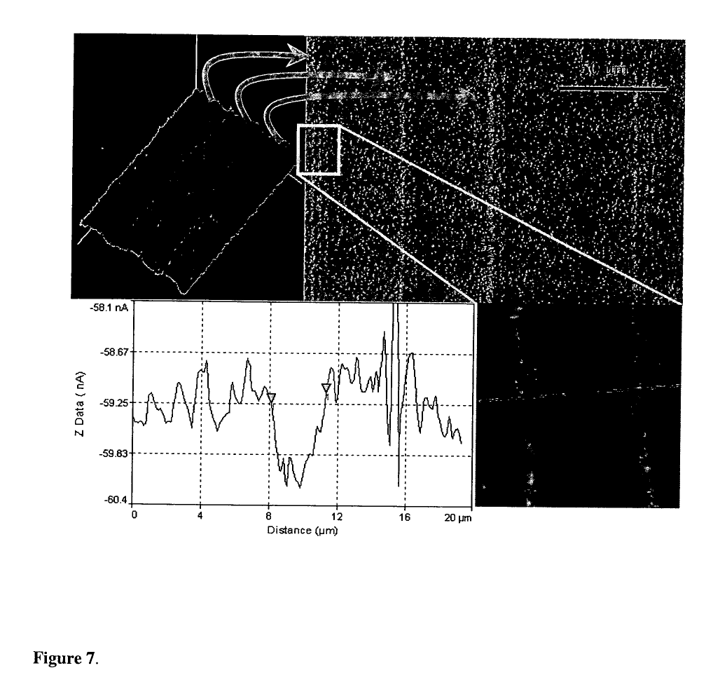

To understand the protein adsorption in ablated channels and compare with the adsorption on rectangular features, the inner surface of the channels were analysed using AFM (FIG. 6). Apart from the hydrophilic elevated ridges observed before, the AFM analysis has revealed a hydrophobic elevated-from-the-bottom-of-the-channel line. The high resolution images of the fluorescence compared with high resolution AFM lateral force mapping (FIG. 7) reveal a ‘fine structure’ of protein deposition, preferentially on the hydrophilic edges of the channel and on the hydrophobic ridge on the center of the channel.

The high specific surface of the channel, which is caused by either the uneven bottom of the channel or by the possible porous material of the ridges, cooperate with the many variations of the surface hydrophobicity to allow a high concentration of diverse proteins in the ablated channels compared with rectangular ablated areas.

### Example 3

### Specific Antibodies Recognition.

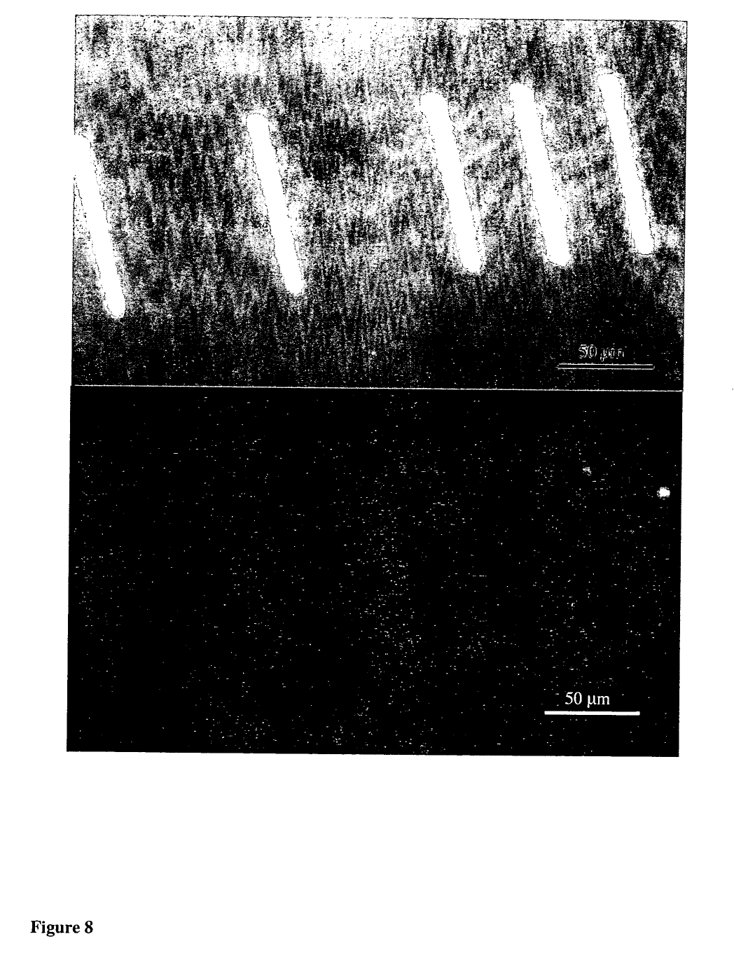

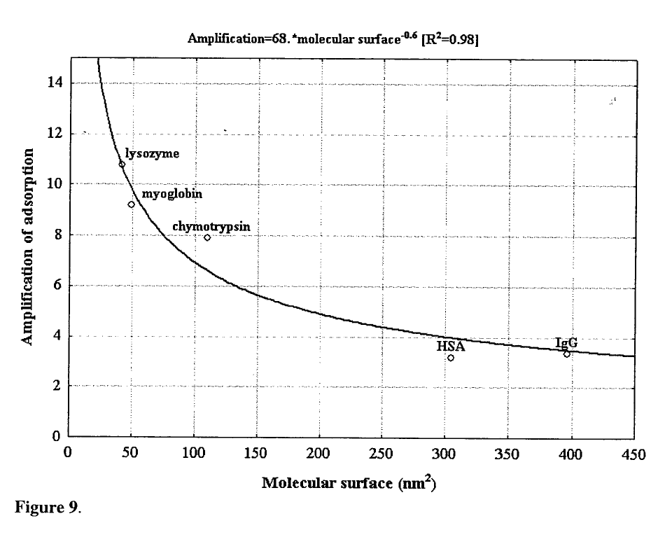

The protein detection system described above was demonstrated by the incubation of the ‘bar code’ assay (fabricated as described above) with both IgG's and control proteins. For an example, FIG. 8 presents a part of an array format with a ‘bar code’ structure that was functionalized with two proteins, before (top image, bright field) and after protein recognition (bottom image, fluorescence). Chicken IgG was deposited on a fragment of the ‘bar code’ structure (three lines on the right, FIG. 8 top) and streptavidin was deposited on the rest of the structure (two lines on the left, FIG. 8 top) using the picoliter pipette. The anti-chicken IgG deposited over the whole array of specifically recognized the chicken IgG lines (bottom image).

### Example 4

### Protein Adsorption

Five, molecularly different proteins on micro/nano-structures fabricated via laser ablation, were used to probe the relationship between the amplification of the protein adsorption and their molecular characteristics (total molecular surface and charge and hydrophobicity-specific surface).

Glass slides or cover slips were thoroughly cleaned, ‘primed’ with hexa-methyl-disilazane via spin coating, then spin-coated with a 4 wt % solution of Poly(methyl methacrylate) -PMMA- in propylene glycol methyl ether acetate at 3000 rpm. The coated substrates were then soft baked at 85° C. for 30 min, and stored in a desiccator prior to and after gold deposition. Gold layers of 50 nm thickness were obtained via the deposition in a sputtering SEM-coating unit E5100 (Polaron Equipment Ltd) at 25 mA for 90 s at 0.1 Torr. The gold-layered substrata were then incubated with bovine serum albumin (BSA) by immersion in a 1% w/v BSA 10 mM phosphate-buffered saline (PBS) solution (pH 7.4) at room temperature for approximately 1 h, and then rinsed with PBS followed by Nanopure water.

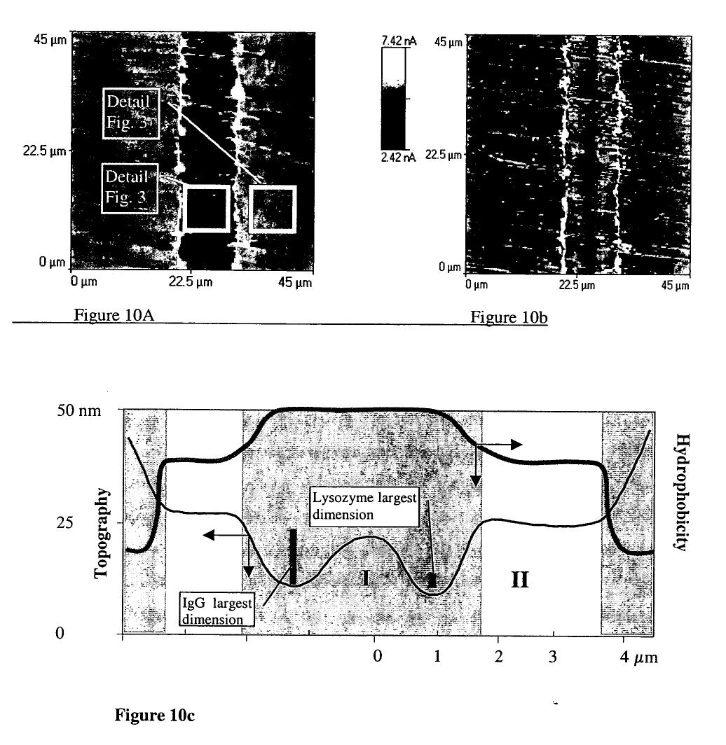

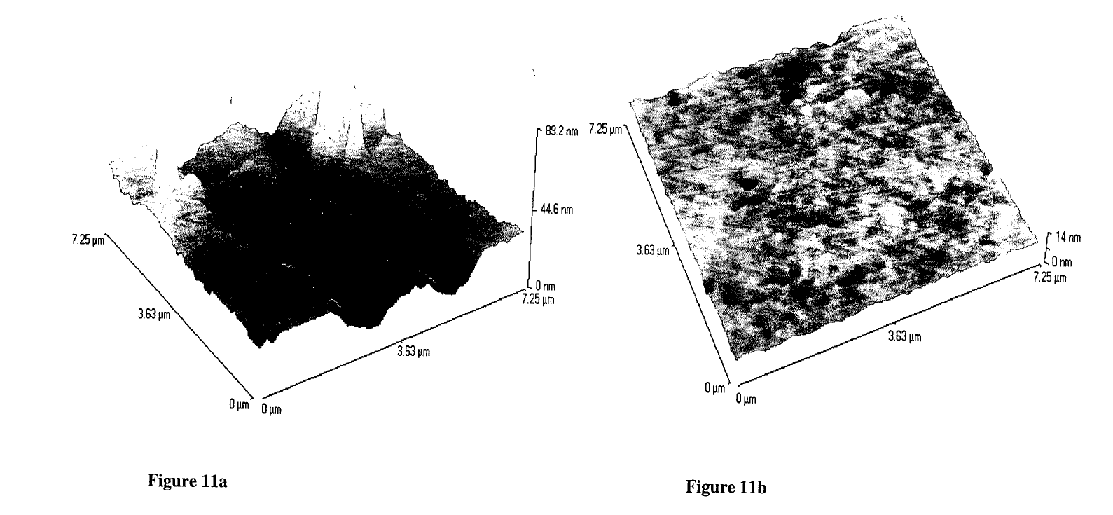

Atomic Force Microscopy (AFM) was carried out on an Explorer system (ThermoMicroscopes) in the normal contact mode. The AFM system is based on detection of tip-to-surface forces through monitoring optical deflection of a laser beam incident on a force-sensing/imposing lever. Several scanners were used in order to cover the scales of lateral topographical and chemical differentiation; the fields-of-view ranged from 100×100 μm down to 8×8 μm. The analyses were carried out under air-ambient conditions (temperature of 23° C. and 45% relative humidity). Pyramidal-tipped, silicon nitride cantilevers with a spring constant of 0.032 N/m were used. As the tip is scanned across the surface, the lateral force acting on the tip manifests itself through a torsional deformation of the lever, which is sensed by the difference signal on the Left-Right signal on the quadrant detector. The difference signal can be plotted as a function of x-y location in the topographical field of view, and the resulted friction force image can then be correlated directly with the topographical image. The micro/nano-topography of the micro-channels is presented in FIG. 10, which also presents the AFM lateral force mapping that validates the mechanism proposed above. The rugosity of the surface (presented in FIGS. 10c and 11a) is also distributed unevenly, with the region outside the valleys and the plateaus (region II in FIG. 10c) being flatter than region I in FIG. 10c).

The attachment of fluorescently labelled proteins on flat and micro-structured surfaces was visualised and analysed using two microscopes. One was the Nikon Microphot FX microscope with a UV light source (Nikon Mercury Lamp, HBO-100 W/2; Nikon C.SHG1 super high pressure mercury lamp power supply) at 100× objective. These images were captured on a Nikon camera (FX-35WA). The second system was a Nikon inverted microscope (Nikon Eclipse TE-DH 100W, 12V) with an attached UV light source (Nikon TE-FM Epi-Fluorescence). Images were captured on a Nikon Charged Coupling Device (CCD) camera.

The fluorescently labeled proteins (10 μl of 70-330 μg/ml) were deposited on flat PMMA and on micro-structured PMMA surfaces (fabricated as described above) flooding the whole surface of the micro-assay. The slide was incubated for 30 min at room temperature in a humid chamber, and then washed three times with PBS and twice with Nanopure water.

The fluorescence intensities were measured with a FluorStar Galaxy Fluor reader (Germany) by measuring emission at 556 nm with excitation at 583 nm. Calibration curves were generated for each protein in order to take into account the degree of labeling. The fluorescence intensities for the proteins deposited on micro-structured surfaces were weighted against the actual area available for deposition (i.e. micron-sized channels), which represents 4% of the total area (0.11 mm²) of deposited droplet of protein solution.

The molecular descriptors of the selected proteins have been calculated using a program, which can be freely downloaded from www.bionanoeng.com, that uses the Connolly algorithm (Connolly, 1973) beyond its original purpose (i.e. the calculation of molecular surface) for the calculation of the surface-related molecular properties (i.e. surface positive and negative charges, surface hydrophobicity and hydrophilicity; the last two using Kyte-Doolittle scale of hydrophobicity/hydrophilicity) as well as the molecular surfaces related to these properties. The program calculates the surface properties using probing balls with different radius. The charges of individual amino acids have been calculated using a semi-empirical method (PM3 as implemented in HyperChem from HyperCube Inc.) for the structures relevant to a particular pH; then averaged according to acid-base equilibria equations; then implemented in an input table read by the program. This procedure allows the calculation of the charges on the protein surface as function of the pH of the solution, and therefore account for the modulation of the adsorption by the differences between the pH and the isoelectric point of the protein. The algorithm used by the program has been reported elsewhere (Cao et al., 2002). The properties of the proteins have been calculated for a radius of the probing sphere between 1.4 Å and 10 Å.

The molecular structures of the selected protein have been imported from the Protein Data Bank (PDB) (Bernstein et al., 1977; Berman et al., 2000). The primary results, which vary importantly with the radius of the probing sphere, are presented in Table 1. The structure and the molecular surface of the selected proteins has been visualised with ViewerLite (from Accelerys).

TABLE 1
Molecular characteristics of the proteins studied calculated for the probing the
protein with a 10 Å radius sphere

| Protein → | | | | Human | |
|---|---|---|---|---|---|
| Descriptor | | | α-Chymo- | serum | |
| ↓ | Lysozyme | Myoglobin | trypsin | albumin | Human IgG |
| PDB code | 1LYZ | 1YMB | 4CHA | 1E78 | 1IGT |
| Molecular | 14000 | 66000 | 24000 | 66500 | 146000 |
| weight | | | | | |
| Size | 42 × 36 × 47 | 46 × 41 × 41 | 59 × 47 × 72 | 129 × 108 × 128 | 109 × 150 × 133 |
| (x, y, z) Å | | | | | |
| Isoelectric | 10.7 | 7.8 | 4.6 | 4.7 | 7.36 |
| point | | | | | |
| Connolly | 4441 | 5197 | 13194 | 30499 | 39418 |
| surface | | | | | |
| area, Å² | | | | | |

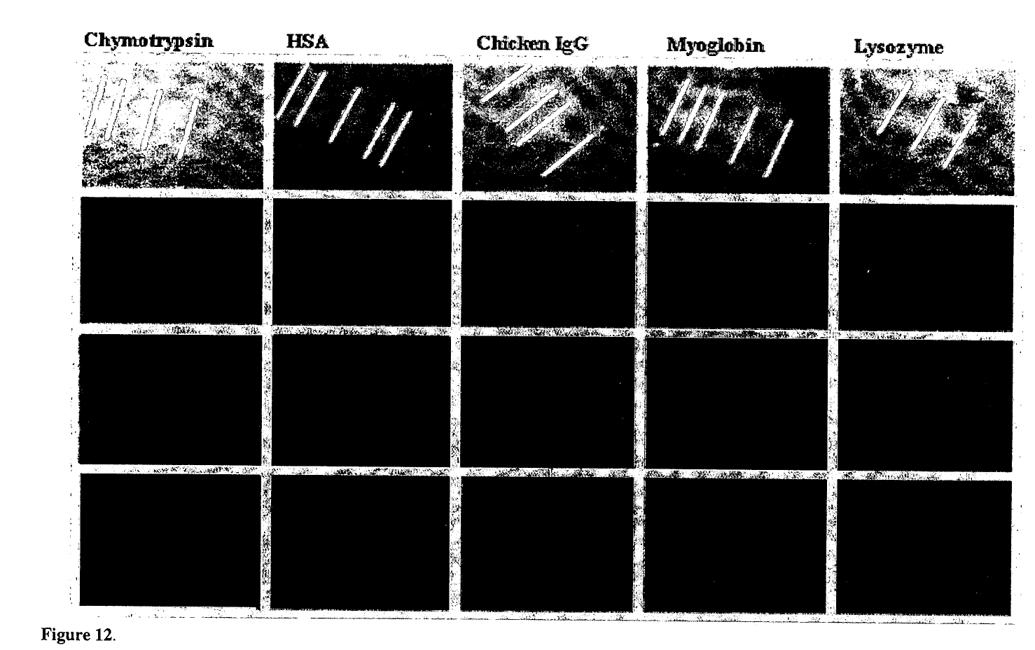

FIG. 12 presents the microscopic images of one set of channels per protein, in bright field mode (the lines are transparent, i.e. white in images in the top row in FIG. 12; whereas the unablated Au layer is opaque); and in fluorescence mode. As expected the higher concentration of the protein the higher the adsorption. However, the sensitivity of the analysis of the impact of molecular descriptors would increase for lower concentrations of proteins. Although in fluorescence mode the adsorption of some proteins could not be visualised with a good contrast, in particular for α-chymotypsin, the photon-counting detection of the Fluor reader could accurately detect variations in protein concentration. Furthermore, the area that is read for the quantification of florescence comprises several areas with bar-code-like lines, which amplifies the read signal. It appears that a concentration of 0.1 mg/ml would allow a high enough level of the fluorescence signal without compromising the sensitivity. This concentration has been reported as optimal, in particular for HSA (Sheller at al., 1998).

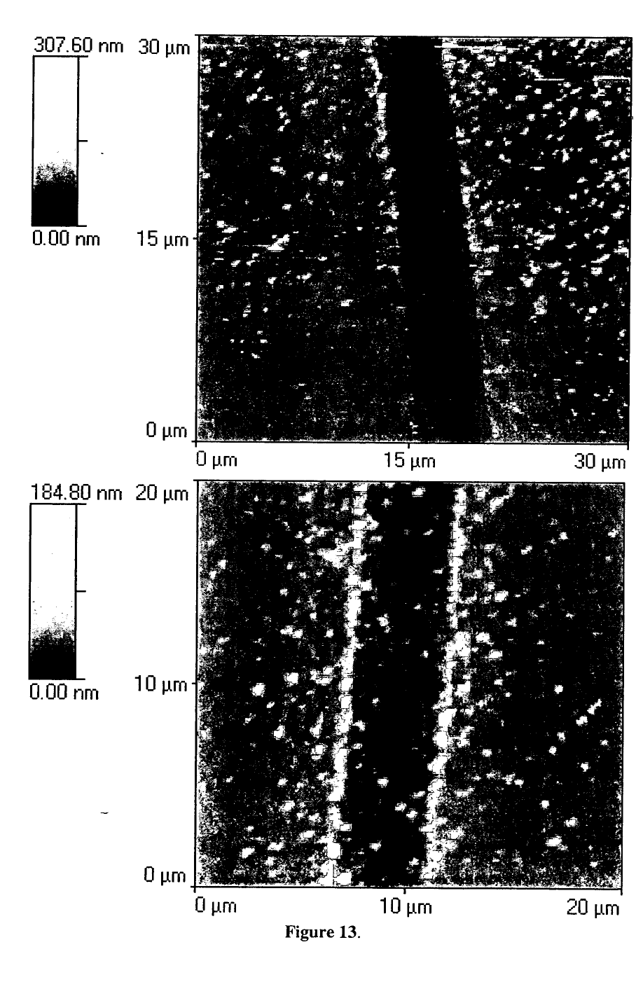

As the micro-structures fabricated as described above comprise micro/nano-areas with very different chemistries it is expected that both hydrophobicity and electrostatics would contribute to the adsorption on these ‘combinatorialised’ micro/nano surface. Indeed an AFM analysis of the topography of the channels after the deposition of proteins (FIG. 13) shows that indeed the initial topography of the channels is partially smoothed, with IgG having more binding at the hydrophobic areas of the channel.

Micro/nano-structures that are micro-sized in width and tens of nanometers deep induce the amplification of protein adsorption 3- to 12-fold depending on the molecular surface of the protein. The smaller proteins can capitalize better on the newly created micro-level structure and nano-level rugosity. The fabrication of the microstructures, the ablation of a thin metallic layer deposited on a non-ablatable PMMA layer, induces the creation of a multitude of surface chemistries, which in turn makes the protein adsorption less dependent on the local molecular descriptors, i.e. hydrophobicity and charges. Consequently, molecularly different proteins will adsorb at increased levels with better chances for the preservation of bioactivity. The amplified and ‘combinatorialised’ adsorption on micro/nano-structures has the potential of improving the detection of biomolecular recognition if used for protein microarrays.

References

Andrade, J. D.; Hiady, V. J. Biomate. Sci. & Polymers 1991, 2, 161-72.

Berman, H. M., Westbrook, J., Feng, Z., Gilliland, G., Bhat, T. N., Weissig, H., Shindyalov, I. N., Bourne, P. E., Nucleic Acid Research, 2000, 28, 235-242.

Bernard, A.; Delamarche, E.; Schmid, H.; Michel, B.; Bosshard, H. R.; Biebuyck, H.; Langmuir 1998, 14, 2225-2229.

Bernstein, F. C., Koetzle, T. F., Williams, G. J. B., Meyer Jr, E. F., Brice, M. D., Rodgers, J. D., Kennard, O., Shimanouchi, T., Tasumi, M., J. Mol. Biol, 1977, 112, 535-542.

Cao, J., Pham, D. K., Tonge, L., Nicolau, D. V., Smart Materials & Structures, 2002, 11(5), 772-777.

Connolly, M. L., J. Mol. Graphics, 1993, 11(2), 139-141.

De Wildt, R. M.; Mundy, C. R.; Gorick, B. D.; Tomlinson, I. M. Nature Biotech. 2000, 18, 989-994.

Fodor, S. P. A.; Read, J. L.; Pirrung, M. C.; Stryer, L.; Lu, A. T.; Solas, D. Science 1991, 251, 767-773.

Ismagilov, R. F.; Ng, J. M.; Kenis, P. J.; Whitesides, G. M. Anal. Chem. 2001, 73, 5207-5213.

Kendrew, J. C., Dickerson, R. E., Strandberg, R. G., Hart, R. G., Davies, D. R., Phillips, D. C., Shore, V. C., Nature, 1960, 155, 422-427.

Levitt, M., Clothia, C., Nature, 1976, 261, 552-558.

McDonald J. C.; Metallo S. J.; Whitesides G. M. Anal. Chem. 2001, 73, 5645-5650.

Nicolau, D. V.; Taguchi, T.; Taniguchi, H.; Yoshikawa, S. Langmuir 1998, 14, 1927-1936.

Nicolau, D. V.; Taguchi, T.; Taniguchi, H.; Yoshikawa, S.; Langmuir 1999, 15, 3845-3851.

Nicolau, D. V.; Cross, R. Biosens. & Bioelectron. 2000, 15, 85-91.

Noy, A.; Vezenov, D. V.; Lieber, C. M. Annu. Rev. Mater. Sci. 1997, 27, 381-421.

Petrash, S., Sheller, N. B., Dando, W., Foster, M. D., Langmuir, 1977, 13, 1881-1883.

Schulz, G. E., Angew. Chem. Int. Ed. Engl., 1977, 16, 23-32.

Schwarz, A.: Rossier, J. S.; Roulet, E.; Mermod, N.; Roberts, V. A.; Girault, R. H. Langmuir 1998, 14, 5526-5531.

Sheller, N. B., Petrash, S., Foster, M. D., Langmuir, 1998, 14, 4535-4544.

Sinniah, S. K., Steel, A. B., Miller, C. J., Reutt-Robey, J. E. J. Am. Chem. Soc. 1996, 118, 8925-8931.

Sundberg, S. A. Curr. Opin. Biotech. 2000, 11, 47-53.

Tsukada, H., Blow, D. M., J. Mol. Biol., 1985, 184, 703.

Vezenov, D. V.; Noy, A.; Rozsnyai, L. F.; Lieber, C. M. J. Am. Chem. Soc. 1997, 119, 2006-2015.

Wadu-Mesthtige, K.; Xu, S.; Amro, N. A.; Liu, G. Langmuir 1999, 15, 8580 -8583.

Walter, G.; Bussow, K.; Cahill, D.; Lueking, A.; Lehrach, H. Curr. Opin. Microbiol. 2000, 3, 298-302.

Wang, C.; Oleschuk, R.; Ouchen, F.; Li, J.; Thibault, P.; Harrison, D. J. Rapid Com. Mass Spectr. 2000, 14, 1377-1383.

Wilbur, J. L.; Biebuyck, H. A.; MacDonald, J. C.; Whitesides, G. M. Langmuir 1995, 11, 825-831.

Zhao, X. M.; xia, Y. N.; Whitesides, G. M. J. Mater. Chem. 1997, 7, 1067-1074.

We claim:

1. A structure comprising
(i) a first layer comprising a molecule-adsorbing, substantially non-ablatable material; and
(ii) a second layer comprising an ablatable material;
wherein the second layer is disposed on the first layer and wherein at least a portion of the second layer has been ablated to expose a surface of the first layer and form at least one profiled feature.

2. The structure according to claim 1, wherein the exposed surface of the first layer comprises at least two localized areas having molecule-adsorbing capacities for molecules with different adsorbing properties.

3. The structure according to claim 1, wherein a plurality of portions of the second layer have been ablated to form a plurality of profiled features.

4. The structure according to claim 3, wherein the plurality of profiled features form an informationally-addressable pattern.

5. The structure according to claim 1, wherein the first layer is a polymeric material.

6. The structure according to claim 5, wherein the exposed surface of the polymeric material has localized areas which have diverse surface properties.

7. The structure according to claim 6, wherein the exposed surface of the polymeric material has localised areas which are hydrophobic, hydrophilic, acidic, basic, charged or neutral.

8. The structure according to claim 1, wherein the first layer comprises a polymeric material selected from the group consisting of polyacrylates, polycarbonates, polystyrenes, fluorine-containing polymers, polyethylenes and derivatives thereof.

9. The structure according to claim 8, wherein the polymeric material is selected from the group consisting of polymethylmethacrylate (PMMA), polyacrylic acid, polyacrylonitrile, polymethacrylate, styrene-acrylonitrile copolymers, butadiene-styrene copolymers, polyalkylstyrenes and polytetrafluoroethylene.

10. The structure according to claim 9, wherein the polymeric material is polymethylmethacrylate.

11. The structure according to claim 7, wherein the localized areas form a predetermined pattern on the surface of the first layer.

12. The structure according to claim 1, wherein the second layer is a metal that can be deposited in a thin layer.

13. The structure according to claim 1, wherein the second layer is a metal selected from the group consisting of Au, Cr, Ag, Mg, Ti, V, Mn, Fe, Co, Ni, Cu, Zn, Cd, Pt, Pd, Rh, Ru, Mo,W and Pb.

14. The structure according to claim 13, wherein the metal is selected from the group consisting of Ag, Cr and Au.

15. The structure according to claim 1, further comprising a blocking layer disposed on the surface of the second layer.

16. The structure according to claim 15, wherein the blocking layer is a material selected from the group consisting of inert polymers, Self Assembled Monolayers, multilayer thin films and inert proteins.

17. The structure according to claim 1, further comprising a substrate that supports the first and second layers.

18. The structure according to claim 17, wherein the substrate is selected from the group consisting of quartz glass, mesoporous silica, nanoporous alumina, ceramic plates, glass, graphite and mica.

19. The structure according to claim 17, wherein the substrate is part of an apparatus for fabricating the structure or for performing an assay.

20. The structure according to claim 1, wherein the profiled feature is a micro-well or a micro-channel.

21. The structure according to claim 20, wherein the micro-wells have a diameter in the range of sub-microns to 50 μm.

22. The structure according to claim 20, wherein the micro-channels have a width in the range of sub-microns to 50 μm.

23. The structure according to claim 20 wherein the micro-channels have a length in the range of 5 to 200 μm.

24. A method of fabricating a structure according to claim 1, comprising the steps of:
(a) obtaining a substrate supporting
(i) a first layer comprising a molecule-adsorbing, substantially non-ablatable material; and
(ii) a second layer comprising an ablatable material disposed on the first layer;
(b) laser ablating at least a portion of the second layer to expose a surface of the first layer to form at least one profiled feature.

25. The method according to claim 24, wherein the first layer is applied to the substrate by sputter coating or spin coating.

26. The method according to claim 25, wherein the first layer is applied to the substrate by spin coating.

27. The method according to claim 24, wherein the second layer is applied to the first layer by sputter coating, spin coating or electroplating.

28. The method according to claim 27, wherein the second layer is applied to the first layer by sputter coating.

29. The method according to claim 24, further comprising the step of applying a blocking layer to the surface of the second layer before laser ablation.

30. The method according to claim 29, wherein the blocking layer is an inert polymer, Self Assembled Monolayer, a multilayer thin film or an inert protein.

31. The method according to claim 30, wherein the inert polymer is applied by spin coating.

32. The method according to claim 30, wherein the Self Assembled Monolayer is formed by immersion of the substrate supporting the first and second layers in a solution of the molecule that forms the Self Assembled Layer.

33. The method according to claim 30, wherein the multilayer thin film is formed by the steps of:
(a) immersion of the substrate supporting the first and second layers in a solution of first polyelectrolyte having a first charge and
(b) immersion of the substrate obtained from (a) in a solution of second polyelectrolyte having a charge complementary to the first polyelectrolyte.

34. The method according to claim 30, wherein the inert protein is applied by immersion and incubation of the substrate supporting the first and second layers in a solution of inert protein, by soaking in an inert protein solution, or by addition of a droplet of a protein solution to the surface of the second layer.

35. The method according to claim 24, wherein the laser ablation is performed with a laser wavelength in the range of 100 nm to 1200 nm.

36. The method according to claim 24, wherein the structure is fabricated on a fabrication platform consisting of a computer controlled laser ablation system comprising a research grade inverted optical microscope, a pulsed nitrogen laser emitting at 337 nm and a programmable XYZ stage.

37. The method according to claim 24, wherein in step (b) a plurality of portions of the second layer are laser ablated to form a plurality of profiled features.

38. The method according to claim 24, wherein the plurality of profiled features are fabricated in an informationally-addressable pattern.

39. An array comprising
(a) a micro-structure which comprises:
(i) a first layer comprising a molecule-adsorbing, substantially non-ablatable material, and
(ii) a second layer of ablatable material;
wherein the second layer is disposed on the first layer and a plurality of portions of the second layer have been ablated to expose a surface of the first layer and thereby form a plurality of profiled features; and
(b) at least one biomolecule adsorbed on the surface of the first layer in at least one of the plurality of profiled features.

40. The array according to claim 39, wherein each of the plurality of profiled features has the same biomolecule adsorbed on the surface of the first layer.

41. The array according to claim 39, wherein each of the plurality of profiled features has a different biomolecule adsorbed on the surface of the first layer.

42. The array according to claim 39, wherein the biomolecule is selected from the group consisting of a gene, DNA, RNA, an oligonucleotide, a protein, a peptide, a polysaccharide, a drug, a potential drug, an antibody, an antigen, an enzyme or an enzyme substrate.

43. A method of preparing an array according to claim 39 comprising
(a) obtaining a structure according to claim 1; and
(b) contacting at least one profiled feature with a biomolecule.

44. The method according to claim 43, wherein the biomolecule is selected from the group consisting of a gene, DNA, RNA, an oligonucleotide, a protein, a peptide, a polysaccharide, a drug, a potential drug, an antibody, an antigen, an enzyme or an enzyme substrate.

45. The method according to claim 43, wherein the biomolecule is contacted with each of the profiled features by flooding the array with a solution containing the biomolecule.

46. The method according to claim 43, wherein the biomolecule is deposited in each of the profiled features using a pico-litre pipette.

47. An assay method comprising the steps of:
(i) contacting an array according to claim 39 with a test sample that may contain an analyte that interacts with the at least one biomolecule adsorbed on the surface of the first layer within at least one profiled feature;
(ii) detecting binding of the analyte to the adsorbed biomolecule.

48. The assay method according to claim 47, wherein the biomolecule and the analyte, in any order, are selected from the group of complementary recognition components consisting of protein and protein, protein and DNA, RNA or oligonucleotide, protein and oligosaccharide or polysaccharide, protein and drug, enzyme and substrate, enzyme and inhibitor; drug and receptor, DNA, RNA or oligonucleotide and their complementary strand, antibody and antigen, DNA, RNA or oligonucleotide and drug, DNA, RNA or oligonucleotide and oligosaccharide or polysaccharide.

49. The assay method according to claim 47, wherein the binding of the analyte and the adsorbed biomolecule is detected by use of fluorescent, phosphorescent, luminescent or radioactive markers or the use of nanoparticles or magnetic beads.

50. The assay method according to claim 47, which is a diagnostic assay method.

51. The assay method according to claim 47, which is a high throughput screening assay method.

52. The assay method according to claim 47, wherein the plurality of profiled features in the array form an informationally-addressable pattern.

53. The assay method according to claim 52, wherein the informationally-addressable pattern encodes information about the biomolecule adsorbed on the exposed surface of the first layer in the plurality of profiled features forming the informationally-addressable pattern.

54. The assay method according to claim 52, wherein the informationally-addressable pattern encodes information about the analyte contacted with the biomolecule adsorbed on the exposed surface of the first layer in the plurality of profiled features forming the informationally-addressable pattern.
